(19) United States
(12) Patent Application Publication
(10) Pub. No.: US 2008/0280323 A1
(43) Pub. Date: Nov. 13, 2008

(54) Method for Producing Epidermal Growth Factor Using Fusion Proteins Comprising Fas-1 Domain

(76) Inventors: Sun Lee, Daejeon Metropolitan City (KR); Jae-Geun Yoo, Daejeon Metropolitan City (KR); In San Kim, Daegu (KR); Eun Hee Bae, Seoul (KR); Dong Sin Lee, Gyeonggi-do (KR)

Correspondence Address: HARNESS, DICKEY & PIERCE, P.L.C., P.O. BOX 828, BLOOMFIELD HILLS, MI, 48303, US

(21) Appl. No.: 11/666,907

(22) PCT Filed: Nov. 3, 2005
(86) PCT No.: PCT/KR05/03696

§ 371 Date: May 3, 2007

(30) Foreign Application Priority Data
Nov. 3, 2004 (KR) 10-2004-0088861

(51) Int. Cl. C12P 21/04; C07K 14/00; C12N 15/11; C07K 1/00; C12N 15/00; A01H 5/00

(52) U.S. Cl. 435/69.1; 530/350; 536/23.4; 435/320.1; 800/298; 530/402

(57) ABSTRACT

The present invention provides a fusion protein in which a polypeptide comprising Fas-1 domain is fused in frame to N-terminal or C-terminal of human EGF, a nucleotide sequence encoding the fusion protein, an expression vector containing the nucleotide sequence, a transformant transformed by the nucleotide sequence and a method for producing human EGF, improving stability of the protein and enhancing the functions of the same. The present invention provides human EGF with improved stability and enhanced functions by fusing a polypeptide comprising Fas-1 domain having the activities of cell adhesion and wound healing to human EGF.

### TECHNICAL FIELD

The present invention relates to a fusion protein and a method for producing the same, more particularly, a polypeptide fusion protein containing human epidermal growth factor (EGF) and Fas-1 domain and a method for producing the same.

### BACKGROUND ART

Epidermal growth factor (EGF) has various functions as follows. EGF can help skin regeneration when it is applied to the ulcerative lesion including diabetic tinea ulcer and decubitus ulcer, so as to prevent the quadruple amputation resulted from the worsening of condition, and is also helpful for the treatment of intractable chronic dermal ulcer and gastric ulcer. In addition, EGF is very effective for skin regeneration of an operated area and for minimizing scar after such operations as keratoplasty and Cesarean section. It also accelerates the regeneration of burned skin, improves wrinkles and can be used as a raw material for anti-aging cosmetics stimulating skin regeneration.

Different attempts have been made to produce epidermal growth factor, so far.

First, there were attempts to produce epidermal growth factor directly from human urine (Gregory et al., Hoppe Seylers Z Physiol Chem., V.356, 1765-74, 1975; Savage et al., Anal Biochem., V.111, 195-202, 1981). However, these methods had problems of low recovery owing to frequent precipitation and concentration processes during purification and inadequacy for mass-production.

Second, human epidermal growth factor gene was expressed in E. coli (Smith et al., Nucleic Acids Res., V.10, 4467-82, 1982; Kishimoto et al., Gene, V.45, 311-6. 1986), Bacillus subtillis (Yamagata et al., Proc Natl Acad Sci USA. V. 86, 3589-93, 1989) and yeast (Urdea et al., Proc Natl Acad Sci USA. V.80, 7461-5, 1983), but when it was expressed in E. coli, it was easily degraded by protease, resulting in low recovery and unsatisfactory gene expression. To obtain human EGF with high degree of purity, various processes including high performance liquid chromatography were required, suggesting another problem of high cost.

Another method for producing EGF was reported, which took advantage of an expression vector secreting EGF out of cells in order to protect EGF from being degraded by protease (Korean Patent No. 102993). Precisely, the method used transformed E. coli in which EGF was secreted out of cells, reducing the endotoxin and contamination by intracellular foreign proteins. However, the method also had problems of high production cost resulted from complicated purification processes, which are the first purification using reverse phase chromatography, the second purification using anion exchange resin and the final purification with reverse phase high performance liquid chromatography using radial compressed C18 column, and stability of EGF as a product in question.

In the meantime, studies on the production of EGF using a fusion protein have been conducted but only with troublesome processes, which comprised the steps of producing a fusion protein, digesting endopeptidase ligated region and purifying EGF therefrom.

This description refers to numbers of patent documents and papers, which are in quotes. The whole contents of the quoted patent documents and papers are supposed to be inserted as reference to describe in more detail the scope and spirit of the present invention.

### DISCLOSURE

### Technical Problem

The present inventors have studied on a method to produce human epidermal growth factor (EGF) effectively and to improve the functions thereof. As a result, the present inventors completed this invention by confirming that human EGF can be produced in a plant in the form of a fusion protein and the selection of a polypeptide containing Fas-1 domain as a fusion partner paves the way to produce EGF with improved functions and stability.

It is an object of the present invention to provide a fusion protein in which a polypeptide containing Fas-1 domain is linked in frame to the direction of N-terminal or C-terminal of human EGF.

It is another object of the present invention to provide a nucleotide sequence encoding the fusion protein.

It is another object of the present invention to provide an expression vector containing the nucleotide sequence.

It is another object of the present invention to provide a transformant transformed by the expression vector.

It is another object of the present invention to provide a method for producing human EGF comprising the steps of preparing a transformant transformed with the expression vector and recovering the expressed protein therefrom.

It is another object of the present invention to provide a method for endowing stability to a peptide or a protein which is characterized by giving stability to a peptide or a protein being unstable when it is expressed after being fused to a polypeptide containing Fas-1 domain.

It is still another object of the present invention to provide a method for improving the functions of human EGF which is characterized by the fusion of a polypeptide containing Fas-1 domain to human EGF.

### Technical Solution

According to an exemplary embodiment of the present invention, the present invention provides a fusion protein in which a polypeptide containing Fas-1 domain is linked in frame to the direction of N-terminal or C-terminal of human EGF.

The present invention is characterized by producing EGF as a fusion protein including EGF and using a polypeptide containing Fas-1 domain as a fusion partner.

Fas-1 domain is a very conserved sequence found in membrane proteins or secretory proteins of many species including mammalia, insect, sea urchin, plant, yeast and bacteria (Kawamoto T. et al., Biochim. Biophys, Acta, 288-292, 1998). Fas-1 domain is composed of approximately 110˜140 amino acids, and particularly contains two very conserved branches (H1 and H2), each of which is composed of 10 highly homologous amino acids (Kawamoto T. et al., Biochim. Biophys, Acta, 288-292, 1998).

A protein harboring Fas-1 domain is exemplified by βig-h3, periostin, fasciclin I, sea urchin HLC-2, algal-CAM and mycobacterium MPB70, etc (Huber, O. et al., EMBOJ., 4212-4222, 1994; Matsumoto, S. et al., J. Immunol., 281-287, 1995; Takeshita, S. et al., Biochem. J., 271-278, 1993; Wang, W. C. et al., J. Biol. Chem., 1448-1455, 1993). Among these proteins, βig-h3, periostin and fasciclin I have 4 Fas-1 domains, respectively, and HLC-2 has two and MPB70 has only one Fas-1 domain.

The biological functions of the proteins harboring Fas-1 domain have not been fully explained, yet some of them have been reported to act as cell adhesion molecules. According to the previous reports, βig-h3 mediates adhesion of fibroblasts and epithelial cells, periostin medicates adhesion of osteoblasts and fasciclin I mediates adhesion of nerve cells (LeBaron, R. G. et al., J. Invest. Dermatol., 844-849, 1995; Horinchi, K. et al., J. Bone Miner. Res., 1239-1249, 1999; Wang, W. C. et al., J. Biol. Chem., 1448-1455, 1993). In addition, algal-CAM was confirmed to be another cell adhesion molecule, being found in embryos of avian volvox (Huber, O. et al., EMBO J., 4212-4222, 1994).

βig-h3 (TGF-β-inducible gene-h3) has a fibrillar structure and interacts with extracellular matrix proteins such as fibronectin and collagen (Kim J.-E., et al., Invest. Opthalmol. Vis. Sci., 43:656-661, 2002). βig-h3 is also known to be involved in cell growth and differentiation, wound healing and morphogenesis (Skonier J., et al., DNA Cell Biol., 13:571-584, 1994; Dieudonne S. C., et al., J. Cell. Biochem., 76:231-243, 1999; Kim J.-E., et al., J. Cell. Biochem., 77:169-178, 2000; Rawe I. M., et al., Invest. Opthalmol. Vis. Sci., 38:893-900, 1997; LeBaron R. G., et al., J. Invest. Dermatol., 104:844-849, 1995). βig-h3 mediates the adhesion of various types of cells including corneal epithelial cells, chondrocytes and fibroblasts (LeBaron R. G., et al., J. Invest. Dermatol., 104:844-849, 1995; Ohno S., et al., Biochim. Biophys. Acta, 1451: 196-205, 1999; Kim J.-E., et al., J. Biol. Chem., 275:30907-30915, 2000).

It was reported that recombinant proteins containing one or more polypeptide harboring βig-h3 derived Fas-1 domain have advantages over natural proteins, which are easy isolation and purification and excellence in cell adhesion, diffusion and proliferation (Bae, J., et al., Bio. Biophy. Res. Comm., 940-948, 2002; Kim, J., et al., J. Biol. Chem., 30907-30915, 2000). The polypeptide harboring βig-h3 derived Fas-1 domain interacts with integrin αvβ3 of endothelial cell and thus has functions of adhering to endothelial cells, inhibiting cell migration and/or proliferation, inducing apoptosis of endothelial cells, inhibiting angiogenesis and/or suppressing tumor cell growth (PCT/KR2004/000851).

Therefore, the present inventors used the polypeptide containing βig-h3 derived Fas-1 domain as a fusion partner of EGF. The fusion of the polypeptide containing Fas-1 domain to EGF protein results in the enhancement of stability of EGF protein and excellent synergy effect produced by the co-work of the polypeptide containing Fas-1 domain having functions of acceleration of fibroblast adhesion, diffusion and proliferation with biologically active EGF.

In the present invention, instead of using a protein which simply helps the isolation, a polypeptide comprising Fas-1 domain which can improve stability and functions is used as a fusion partner, by which a fusion partner does not need to be cut and can be used as it is.

According to a preferable embodiment of the present invention, the polypeptide containing Fas-1 domain is linked in frame to N-terminal or C-terminal of EGF.

According to another preferable embodiment of the present invention, there are certain numbers of peptides in between Fas-1 domain of a fusion protein and human EGF. The peptide can be inserted for the purpose of isolating human EGF from a fusion protein. To do so, enterokinase digestive site, thrombin digestive site or protease digestive site can be inserted.

The term “βig-h3 (Fas-1 domain)-EGF” herein means that a polypeptide comprising Fas-1 domain of βig-h3 is linked to N-terminal of EGF, and the term “EGF-βig h3 (Fas-1 domain)” means that a polypeptide comprising Fas-1 domain of βig-h3 is linked to C-terminal of EGF.

If not described otherwise, βig-h3 (Fas-1 domain)-EGF is represented by βig-h3-EGF in short and EGF-βig-h3 (Fas-1 domain) is represented by EGF-βig-h3.

In the present invention, the Fas-1 domain of “polypeptide containing Fas-1 domain” is originated from mammalia, preferably derived from a group consisting of human, rat and mouse. Every Fas-1 domain originated from the conventional proteins containing Fas-1 domain can be used in the present invention. That is, every Fas-1 domain detected from NCBI Entrez (http://www.ncbi.nlm.nih.gov/Entrez/) or EMBL-EBI (http://www.ebi.ac.uk/), the general protein sequence search database well known to those skilled in the art, can be used herein.

The Fas-1 domain used in the present invention is originated preferably from βig-h3, periostin, fasciclin I, sea urchin HLC-2, algal-CAM and mycobacterium MPB70, more preferably from Fas-1 domain I, II, III or IV existing in βig-h3, and most preferably from Fas-1 domain II or IV of βig-h3.

According to a preferable embodiment of the invention, a polypeptide comprising Fas-1 domain can include one or more Fas-1 domains.

According to an aspect of the present invention, there is provided a nucleotide sequence encoding a fusion protein fused to a polypeptide containing Fas-1 domain linked to N-terminal or C-terminal of human EGF.

According to an aspect of the present invention, there is also provided an expression vector containing the nucleotide sequence encoding the fusion protein fused in frame to the polypeptide comprising Fas-1 domain linked to N-terminal or C-terminal of human EGF.

According to an aspect of the present invention, there is further provided a transformant transfected with the nucleotide sequence encoding the fusion protein fused in frame to the polypeptide comprising Fas-1 domain linked to N-terminal or C-terminal of human EGF.

According to a preferable embodiment of the present invention, the nucleotide sequence of a polypeptide comprising Fas-1 domain is linked in frame to N-terminal or C-terminal of EGF nucleotide sequence.

The nucleotide sequence of a polypeptide comprising Fas-1 domain used for fusion in the present invention is originated from mammalia, preferably derived from a group consisting of human, rat and mouse. Every nucleotide of Fas-1 domain known to be originated from the conventional proteins containing Fas-1 domain can be used herein. That is, every Fas-1 domain detected from NCBI Entrez (http://www.ncbi.nlm.nih.gov/Entrez/) or EMBL-EBI (http://www.ebi.ac.uk/), the general protein sequence search database well known to those skilled in the art, can be used herein.

According to a preferable embodiment of the present invention, the nucleotide sequence encoding Fas-1 domain used in the present invention is the nucleotide sequence encoding the Fas-1 domain originated from βig-h3, periostin, fasciclin I, sea urchin HLC-2, algal-CAM and mycobacterium MPB70, preferably the nucleotide sequence encoding βig-h3 Fas-1 domain I, II, III or IV, more preferably the nucleotide sequence encoding βig-h3 Fas-1 domain II or IV, and most preferably the nucleotide sequence represented by SEQ. ID. No 2 or No 3, which are modified to be expressed in plants.

According to a preferable embodiment of the present invention, every nucleotide sequence that is able to encode human EGF can be used in the invention, and the nucleotide sequence represented by SEQ. ID. No 1 which is modified for being expressed in plants is preferred.

The nucleotide sequences each represented by SEQ. ID. No 1, No 2 and No 3 are synthesized by using codon commonly applicable to prokaryotic cells, eukaryotic cells and plant cells, based on the amino acid sequences of EGF (Gregory et al., Hoppe Seylers Z Physiol Chem., V.356, 1765-74, 1975) and human βig-h3 (Genbank No. M77349), and another DNA applicable for the synthesis can be additionally used.

As described hereinbefore, the polynucleotide of the present invention is designed to have the optimum sequence for the expression in plants based on the following criterion: (i) adjust GC content to be more than 50%, (ii) contain codons preferred by plants, (iii) eliminate intron-like sequence.

The synthesis of fusion nucleotide sequence and the construction of a vector containing the sequence are performed by the conventional method well-known to those in this field.

According to a preferable embodiment of the present invention, the expression vector of the invention is the vector suitable for the expression in plants, animals or microorganisms. For the vector, the general vector which has been used for the expression of a foreign protein in plants, animals or microorganisms can be used. The vector systems can be constructed by the conventional method well-known to those in this field, and specific processes of the method are described in a reference (Sambrook et al., Molecular Cloning, A Laboratory Manuel, Cold Spring Harbor Laboratory Press (2001)).

The vector of the present invention is constructed by using prokaryotic cells or eukaryotic cells as a host. For example, if a vector is an expression vector and the vector uses prokaryotic cells as a host, it is generally required to contain a strong promoter (Ex: p_{L}^{λ} promoter, trp Promoter, lac promoter, tac promoter, T7 promoter, etc) for transcription, ribosome binding site for the initiation of translation and sequence for the termination of transcription/translation. When E. coli (Ex: HB101, BL21, DH5α, etc) is used as a host cell, promoter and operator region for E. coli tryptophan biosynthetic pathway (Yanofsky, C., J. Bacteriol., 158:1018-1024 (1984)) and left promoter of phage λ (p_{L}^{λ} promoter, Herskowitz, I. and Hagen, D., Ann. Rev. Genet., 14:399-445(1960)) are used as a regulatory region. When bacillus is used as a host cell, a promoter of Bacillus thuringiensis toxoprotein gene (Appl. Environ. Microbiol. 64:3932-3938 (1998); Mol. Gen. Genet. 250:734-741 (1996)) or any other promoter suitable for the expression in bacillus can be used as a regulatory region. In the meantime, a vector for the present invention can be constructed by manipulating plasmids (Ex: pSC101, pGV1106, pACYC177, ColE1, pKT230, pME290, pBR322, pUC8/9, pUC6, pBD9, pHC79, pIJ61, pLAFR1, pHV14, pGEX series, pET series and pUC19), phages (Ex: λgt4·λB, λ-Charon, λΔz1 and M13) or viruses (Ex: SV40) generally used in this field.

If a vector of the invention is an expression vector and uses eukaryotic cells as a host, replication origin operating in eukaryotic cells can be one of f1 replication origin, SV40 replication origin, pMB1 replication origin, Adeno replication origin, AAV replication origin and BBV replication origin, but not always limited thereto. A promoter originated from the genome of mammalian cells (Ex: metallothionine promoter) or a promoter originated from mammalian virus (Ex: adenovirus late promoter, vacinia virus 7.5K promoter, SV40 promoter, cytomegalovirus promoter and HSV tk promoter) can also be used, which have polyadenylated sequence as a transcription terminator sequence. An expression vector for eukaryotic cells, applicable for the present invention, can be selected from various vectors well-known in this field. For example, it can be selected from a group consisting of Ylp5, YCpl9, adenovirus-originated vector, poliovirus-originated vector and bovine papilloma virus-originated vector, etc.

The expression vector used in the present invention includes an antibiotic resistant gene, as a selection marker, which is one of those generally used in this field and exemplified by ampicillin, gentamycin, carbenicillin, chloramphenicol, streptomycin, kanamycin, geneticin, neomycin and tetracycline resistant genes. Among those genes, ampicillin or gentamycin resistant gene is preferred in the point of reducing expenses.

Any host cell informed to this field, suitable for serial cloning and stable expression of a vector, can be used for the present invention, which is exemplified by E. coli JM109, E. coli BL21, E. coli RR1, E. coli LE392, E. coli B, E. coli X 1776, E. coli W3110, Bacillus sp. strain such as Bacillus subtilis and Bacillus thuringiensis, Enterobacter strain such as Salmonella typhimurium, Serratia marcescens and Pseudomonas sp., etc.

If eukaryotic cells are used as a host for the vector of the present invention, yeast (Saccharomyce cerevisiae), insect cells, plant cells and animal cells (Ex: CHO (Chinese hamster ovary), W138, BHK, COS-7, 293, HepG2, 3T3, RIN and MDCK cell lines) can be used as host cells.

If host cells are prokaryotic cells, a vector of the invention can be carried in the host cells by CaCl₂ method (Cohen, S, N. et al., Proc. Natl. Acac. Sci. USA, 9:2110-2114 (1973)), Hanahan's method (Cohen, S, N. et al., Proc. Natl. Acac. Sci. USA, 9:2110-2114 (1973); Hanahan, D., J. Mol. Biol., 166:557-580 (1983)) or electroporation (Dower, W. J. et al., Nucleic. Acids Res., 16:6127-6145 (1988)). If host cells are eukaryotic cells, a vector of the invention can be injected to the host cells by microinjection (Capecchi, M. R., Cell, 22:479 (1980)), calcium phosphate precipitation (Graham, F. L. et al., Virology, 52:456 (1973)), electroporation (Neumann, E. et al., EMBO J., 1:841 (1982)), liposome-mediated transfection (Wong, T. K. et al., Gene, 10:87 (1980)), DEAE-dextran treatment (Gopal, Mol. Cell. Biol., 5:1188-1190 (1985)), or gene bombardment (Yang et al., Proc. Natl. Acad. Sci., 87:9568-9572 (1990)), etc.

For the selection of transformed host cells, phenotype expressed by the mentioned selection marker is observed. For example, if the selection marker is an antibiotic specific resistant gene, a transformant can be easily selected from the culture of cells on a medium containing the specific antibiotics.

According to a preferable embodiment of the present invention, to express an expression vector in E. coli, the nucleotide sequence of the invention was inserted in pET28a (Novagen) expression vector, in which the sequence was in framed to 6 consecutive histidine residues. By the above manipulation, the fused gene was effectively expressed by tac promoter and lac I repressor, leading to the successful isolation of expressed fusion protein and confirmation of functions of the novel EGF.

According to a preferable embodiment of the present invention, a preferred expression vector for invention is a vector for plant and a preferred transformant is also a plant transformant. The expression vector for plant and the method of producing the plant transformant of the invention can be one of the conventional vectors and methods well informed in this field, and in particular, a vector and a method described in PCT/KR02/01461, PCT/KR02/01462 and PCT/KR02/01463 regarding a method for transformation of a specific plant (Ex: tobacco, melon, cucumber, watermelon and rape) can be used. These patent documents are included in this description as references.

According to a preferable embodiment of the present invention, Agrobacterium tumefaciens GV3101 is preferably selected for the transformation of a plant.

According to another aspect of the present invention, there is provided a method for producing human EGF comprising the steps of preparing a transformant transfected with a fusion expression vector containing nucleotide sequence encoding polypeptide comprising Fas-1 domain and a fusion protein of human EGF and recovering the expressed protein therefrom.

The method for producing human EGF of the present invention uses a transformant transformed by a fusion vector having a nucleotide sequence encoding a polypeptide comprising Fas-1 domain and human EGF fusion protein, as explained hereinbefore, so double description is avoided not to be complicated.

According to a preferable embodiment of the present invention, human EGF protein is obtained as it is fused to Fas-1 domain, and if necessary, Fas-1 domain therein can be eliminated. For example, if enterokinase digestive region or thrombin digestive region is in-between Fas-1 domain and human EGF fusion protein, Fas-1 domain can be eliminated by a proper protease.

In the present invention, a protein can be obtained after the conventional purification process. For example, solubility fractionation using ammonium sulfate or PEG, ultrafiltration according to molecular weight, and various chromatographies (designed for the isolation according to size, electric charge, hydrophobicity or affinity) can be used singly or combined to purify a target protein.

According to a preferred embodiment of the present invention, a fusion protein can be prepared by using histidine residue or GST which is generally used for the purification of an expressed protein.

According to an aspect of the present invention, there is also provided a method for improving stability of a peptide or a protein which is apt to be unstable when being expressed as it is fused to a polypeptide comprising Fas-1 domain.

According to a preferred embodiment of the present invention, a peptide or a protein fused to a polypeptide comprising Fas-1 domain is a EGF peptide or protein of human EGF.

A protein fused to a polypeptide comprising Fas-1 domain shows improved stability according to the present invention (see FIG. 14).

According to an aspect of the present invention, there is further provided a method for enhancing the functions of EGF by supplementing the functions of cell adhesion, cell diffusion, cell proliferation and wound healing when a peptide or a protein is fused to a polypeptide comprising Fas-1 domain.

The polypeptide containing Fas-1 domain and EGF fusion protein of the present invention were confirmed to have excellent activities of cell adhesion, cell diffusion and cell proliferation (see FIGS. 11, 13 and 14) in addition to the function of effective wound healing, owing to the presence of a polypeptide comprising Fas-1 domain, and further provide synergy effect generated from the co-work of human EGF and Fas-1 domain.

### Advantageous Effects

As described in detail hereinbefore, the present invention provides a fusion protein in which a polypeptide comprising Fas-1 domain is fused in frame to N-terminal or C-terminal of human EGF, a nucleotide sequence encoding the fusion protein, an expression vector containing the nucleotide sequence, a transformant transformed with the nucleotide sequence and a method for enhancing the production, stability and functions of human EGF using the same. The human EGF of the present invention has improved stability and enhanced functions by the fusion with a polypeptide comprising Fas-1 domain, which is capable of increasing cell adhesion and wound healing.

### DESCRIPTION OF DRAWINGS

FIG. 1 is a schematic diagram showing the cloning process of βig-h3 (Fas-1 domain)-EGF;

FIG. 2 is a schematic diagram showing the cloning process of EGF-βig-h3 (Fas-1 domain);

FIG. 3 is a schematic diagram showing the structure of a recombinant plasmid in which βig-h3 (Fas-1 domain)-EGF or EGF-βig-h3 (Fas-1 domain) fusion gene is inserted;

FIG. 4 is a photograph showing the result of that cells were homogenized and proteins in supernatant were eletrophorezed on polyacrylamide gel;

FIG. 5 is a photograph showing the result of electrophoresis performed on polyacrylamide gel with isolated βig-h3 (Fas-1 domain)-EGF or EGF-βig-h3 (Fas-1 domain) fusion protein;

FIG. 6 is a photograph showing the result of Western blot analysis confirming the fusion protein by using EGF antibody;

FIG. 7 is a schematic diagram showing the structure of a vector for expressing EGF-βig-h3 (Fas-1 domain) protein gene in a plant;

FIG. 8 is a photograph showing the result of electrophoresis on agarose gel with PCR product of EGF-ig-h3 (Fas-1 domain) fusion gene inserted in each transformant plant;

FIG. 9 is a photograph showing the result of electrophoresis on agarose gel with PCR product of βig-h3 (Fas-1 domain)-EGF fusion gene inserted in each transformant plant;

FIG. 10 is a photograph showing the result of Western blot analysis confirming the fusion protein by using EGF antibody;

FIG. 11 is a graph showing the cell adhesion activities of the fusion proteins of the present invention (Regenin (βig-h3 domain IV), NR1 (EGF-Regenin), NR2 (his tag EGF-Regenin), NR3 (Regenin-EGF), NR4 (EGF-albumin));

FIG. 12 is a graph showing the cell diffusion activities of the fusion proteins of the present invention (Regenin (βig-h3 domain IV), NR1 (EGF-Regenin), NR2 (his tag EGF-Regenin), NR3 (Regenin-EGF), NR4 (EGF-albumin));

FIG. 13 is a graph showing the cell proliferation activities of the fusion proteins of the present invention (Regenin (βig-h3 domain IV), NR1 (EGF-Regenin), NR2 (his tag EGF-Regenin), NR3 (Regenin-EGF), NR4 (EGF-albumin)); and

FIG. 14 is a graph showing the stability of the fusion protein of the present invention (Regenin (βig-h3 domain IV)).

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Synthesis of a Novel EGF Nucleotide Sequence

An optimum nucleotide sequence preferred by a plant was designed, which was different from the conventional nucleotide sequence encoding hEGF but was able to encode 53 amino acids (MNSDSECPLSHDGYCLHDGVCMYIEALDKYACNCVVGYIGERCQYRDLKWWELR) equal to the natural hEGF. It was considered for the design of the novel gene that GC content had to be more than 50%, a codon preferred by a plant had to be included and intron-like sequence had to be eliminated. The resultant nucleotide sequence was synthesized at Plant Biotechnology Institute (referred as “PBI” hereinafter), National Research Centre (SK, Canada). The synthetic DNA encoding hEGF is represented by SEQ. ID. No 1.

### Example 2

### Synthesis of a Novel βig-h3 Fas-1 Domain II Nucleotide Sequence

An optimum nucleotide sequence preferred by a plant was designed, which was different from the conventional nucleotide sequence encoding Fas-1 domain II but was able to encode the nucleotide sequence encoding amino acid sequence containing natural human βig-h3 fas-1 domain II (TNNIQQIIEI EDTFETLRAA VAASGLNTML EGNGQYTLLA PTNEAFEKIP SETLNRILG DPEALRDLLN NHILKSAMCA EAIVAGLSVE TLEGTTLEVG CSGDMLTING KAIISNKDIL ATNGVIHYID ELL). It was considered for the synthesis of the novel gene that GC content had to be more than 50%, a codon preferred by a plant had to be included and intron-like sequence had to be eliminated. The novel nucleotide sequence was chemically synthesized at Plant Biotechnology Institute (referred as “PBI” hereinafter), National Research Centre (SK, Canada). The synthetic DNA encoding fas-1 domain II is represented by SEQ. ID. No 2.

### Example 3

### Synthesis of a Novel βig-h3 Fas-1 Domain IV Nucleotide Sequence

An optimum nucleotide sequence preferred by a plant was designed, which was different from the conventional nucleotide sequence encoding Fas-1 domain IV but was able to encode the nucleotide sequence encoding amino acid sequence containing natural human βig-h3 fas-1 domain IV (MKETAAAKFEREHMDSPDLGTLVPRGSMADILTPPMGTVMDVLKGDNRFSMLVAAIQ SAGLTETLNREGVYTVFAPTNEAFRALPPRERSRLLGDAKELANILKYHIGDEILVSG GIGALVRLKSLQGDKLEVSLKNNVVSVNKEPVAEPDIMATNGVVHVITNVLQPPANLE). It was considered for the synthesis of the novel gene that GC content had to be more than 50%, a codon preferred by a plant had to be included and intron-like sequence had to be eliminated. The novel nucleotide sequence was chemically synthesized at Plant Biotechnology Institute (referred as “PBI” hereinafter), National Research Centre (SK, Canada). The synthetic DNA encoding fas-1 domain IV is represented by SEQ. ID. No 3.

### Example 4

### Preparation of βig-h3-EGF Fusion Nucleotide Sequences

To prepare βig-h3-EGF fusion nucleotide sequence and to insert the sequence into pET28a expression vector, following experiments were performed.

### <4-1> Preparation of EGF/pUC18

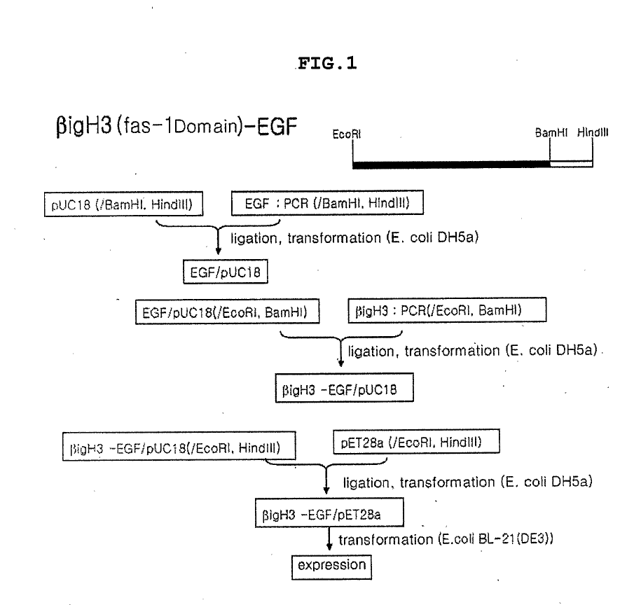

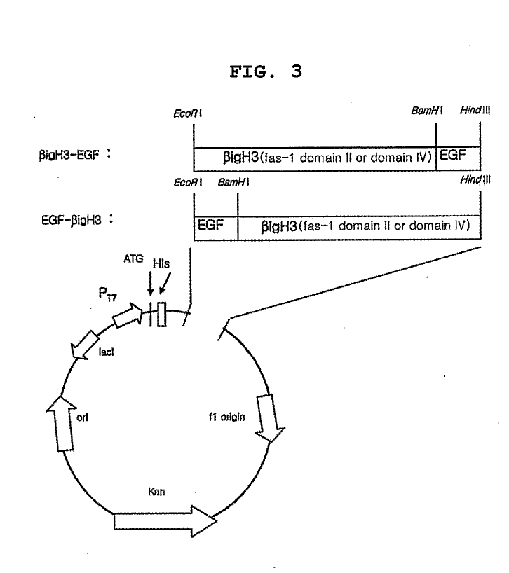

PCR was performed with a set of primers designed to have BamHI recognition site in 5′ end and HindIII recognition site in 3′ end of the synthetic nucleotide sequence encoding EGF of Example 1 by using the synthetic EGF nucleotide sequence as a template (predenaturation at 96° C. for 2 minutes, denaturation at 94° C. for 1 minute, annealing at 55° C. for 1 minute, polymerization at 72° C. for 2 minute, 35 cycles from denaturation to polymerization, and final extension at 72° C. for 10 minutes). The PCR product was treated with restriction enzymes BamHI and HindIII, followed by extraction. The extracted and purified EGF nucleotide sequence was mixed with pUC18 plasmid pre-treated with restriction enzymes BamHI and HindIII (Gibco, BRL, USA) at the ratio of 10:1. Ligation buffer and T4 DNA ligase were added to the mixture solution, followed by reaction at room temperature for one hour. Then, CaCl₂ treated E. coli DH5α (Invitrogen, USA) was transformed by the reactant, followed by selection of strains showing ampicillin resistance from ampicillin (100 mg/ml) containing LB medium. Plasmids were separated from the strains and among them, those plasmids including EGF nucleotide sequence were selected, resulting in the preparation of EGF/pUC18 (see FIG. 1 and FIG. 3).

<4-2> Preparation of βig-h3 (Fas-1 domain)-EGF/pUC18

PCR was performed with a set of primers designed to have EcoRI and BamHI recognition sites respectively in 5′ end and 3′ end of βig-h3 Fas-1 domain II DNA by using βig-h3-Fas-1 domain II DNA synthesized in Example 2 as a template. The PCR product was treated with restriction enzymes EcoRI and BamHI, followed by extraction. PCR was performed once again with a set of primers designed to have EcoRI and BamHI recognition sites respectively in 5′ end and 3′ end of βig-h3-Fas-1 domain IV DNA by using βig-h3-Fas-1 domain IV DNA synthesized in Example 3 as a template. Then, the PCR product was treated with restriction enzymes EcoRI and BamHI, followed by extraction. The extracted and purified nucleotide sequences of βig-h3-Fas-1 domain II and βig-h3-Fas-1 domain IV were mixed with EGF/pUC18 plasmid of Example <4-1>, which had been digested with EcoRI and BamHI, at a proper ratio. Each mixture was ligated by the same manner as described in Example <4-1>, CaCl₂ treated E. coli DH5α was transformed, and ampicillin resistant strains were selected from ampicillin containing (100 mg/ml) LB medium. Plasmids were separated from the strains and those plasmids containing βig-h3 (Fas-1 domain)-EGF fusion nucleotide sequence were selected, resulting in the preparation of βig-h3 (Fas-1 domain)-EGF/pUC18 (see FIG. 1 and FIG. 3).

### Example 4-3

### Preparation of βig-h3 (Fas-1 Domain)-EGF/pET28a

To insert the fusion nucleotide sequence of βig-h3 (Fas-1 domain)-EGF in pET28a expression vector, βig-h3 (Fas-1 domain)-EGF/pUC18 plasmids were digested with restriction enzymes EcoRI and HindIII, followed by electrophoresis to isolate and purify the fusion nucleotide sequence of βig-h3 (Fas-1 domain)-EGF on agarose gel. The fusion nucleotide sequence of βig-h3 (Fas-1 domain)-EGF was mixed with pET28a (Invitrogen, USA) which had been digested with restriction enzymes EcoRI and HindIII (Invitrogen, USA), followed by ligation by the same manner as described in Example <4-1>. Upon transfection of CaCl₂ treated E. coli BL21 (DE3) (Invitrogen, USA), kanamycin resistant strains were selected from kanamycin (100 mg/ml) containing LB medium. Plasmids were isolated from the strains, and those plasmids having fusion nucleotide sequence of βig-h3 (Fas-1 domain)-EGF were selected, resulting in the preparation of βig-h3 (Fas-1 domain)-EGF/pET28a (see FIG. 1 and FIG. 3).

It was confirmed by DNA sequencing that the nucleotide sequence of EGF was successfully fused in frame to the fusion partner βig-h3 (Fas-1 domain).

### Example 5

### Preparation of Fusion Nucleotide Sequence of EGF-βig-h3 (Fas-1 Domain)

To prepare fusion nucleotide sequence of EGF-βig-h3 and to insert the sequence in pET28a expression vector, following experiments were performed.

### <5-1> Preparation of EGF/pUC18

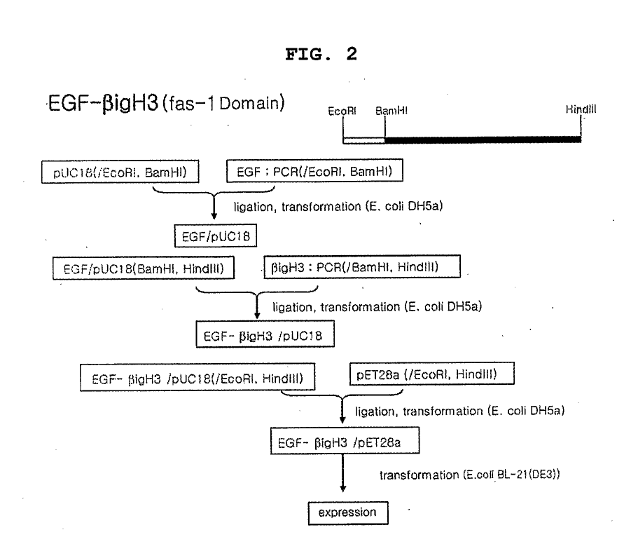

PCR was performed with a set of primers designed to have EcoRI recognition site in 5′ end and BamHI recognition site in 3′ end of the synthetic nucleotide sequence encoding EGF of Example 1 by using the synthetic EGF nucleotide sequence as a template (predenaturation at 96° C. for 2 minutes, denaturation at 94° C. for 1 minute, annealing at 55° C. for 1 minute, polymerization at 72° C. for 2 minute, 35 cycles from denaturation to polymerization, and final extension at 72° C. for 10 minutes). The PCR product was treated with restriction enzymes EcoRI and BamHI, followed by extraction. The extracted and purified EGF nucleotide sequence was mixed with pUC18 plasmid pre-treated with restriction enzymes EcoRI and BamHI at a proper ratio. E. coli DH5α was transformed by the same manner as described in Example <4-1> and ampicillin resistant strains were selected from ampicillin (100 mg/ml) containing LB medium. Plasmids were separated from the strains and among them, those plasmids including EGF nucleotide sequence were selected, resulting in the preparation of EGF/pUC18 (see FIG. 2 and FIG. 3).

<5-2> Preparation of EGF-βig-h3/pUC18

PCR was performed with a set of primers designed to have BamHI and HindIII recognition sites respectively in 5′ end and 3′ end of βig-h3 Fas-1 domain II DNA by using ig-h3-Fas-1 domain II DNA synthesized in Example 2 as a template. The PCR product was treated with restriction enzymes BamHI and HindIII, followed by extraction. PCR was performed once again with a set of primers designed to have BamHI and HindIII recognition sites respectively in 5′ end and 3′ end of βig-h3-Fas-1 domain IV DNA by using βig-h3-Fas-1 domain IV DNA synthesized above as a template. Then, the PCR product was treated with restriction enzymes BamHI and HindIII, followed by extraction. The extracted and purified nucleotide sequences of βig-h3-Fas-1 domain II and βig-h3-Fas-1 domain IV were mixed with EGF/pUC18 plasmid of Example <5-1>, which had been digested with BamHI and HindIII, at a proper ratio. Each mixture was ligated by the same manner as described in Example <4-1>, CaCl₂ treated E. coli DH5α was transformed, and ampicillin resistant strains were selected from ampicillin containing (100 mg/ml) LB medium. Plasmids were separated from the strains and those plasmids containing EGF-βig-h3 fusion nucleotide sequence were selected, resulting in the preparation of EGF-βig-h3/pUC18 (see FIG. 2 and FIG. 3).

<5-3> Preparation of EGF-βig-h3/pET28a

To insert the fusion nucleotide sequence of EGF-βig-h3 in pET28a expression vector, EGF-βig-h3/pUC18 plasmids were digested with restriction enzymes EcoRI and HindIII, followed by electrophoresis to isolate and purify the fusion nucleotide sequence of EGF-βig-h3 on agarose gel. The fusion nucleotide sequence of EGF-βig-h3 was mixed with pET28a which had been digested with restriction enzymes EcoRI and HindIII, followed by ligation by the same manner as described in Example <4-1>. Upon transfection of CaCl₂ treated E. coli BL21 (DE3), kanamycin resistant strains were selected from kanamycin (100 mg/ml) containing LB medium. Plasmids were isolated from the strains, and those plasmids having fusion nucleotide sequence of βig-h3-EGF were selected, resulting in the preparation of EGF-βig-h3/pET28a (see FIG. 2 and FIG. 3).

It was confirmed by DNA sequencing that the nucleotide sequence of EGF was successfully fused in frame to the fusion partner βig-h3.

### Example 6

### Expression of Fusion Protein

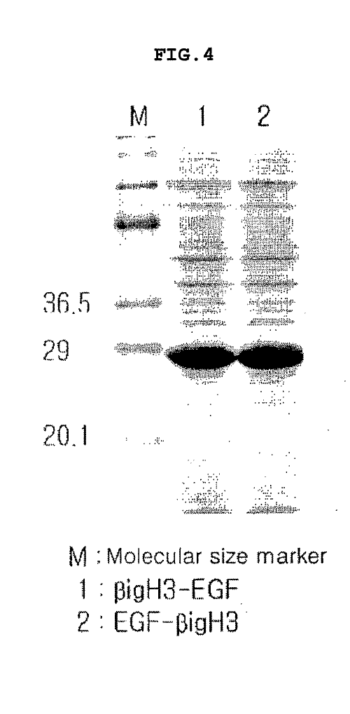

E. coli containing either EGF-βig-h3/pET28α or βig-h3-EGF/pET28a, selected in Example 5, was cultured in a 5 t fermentor until OD₆₅₀ reached 0.5, to which IPTG (0.5 mM) was added in order to induce expression of fusion nucleotide sequence. The culture continued for 5-6 more hours. Then, cells were recovered by centrifugation. The recovered cells was completely suspended in 40 ml of buffer solution (50 mM Tris, pH 8.0, 1 in M EDTA). Cells were disrupted by ultrasonicator, and then supernatant was separated by centrifugation. The supernatant was analyzed by 12% polyacrylamide gel electrophoresis to investigate the expression of fusion protein according as the expression is induced or not (see FIG. 4).

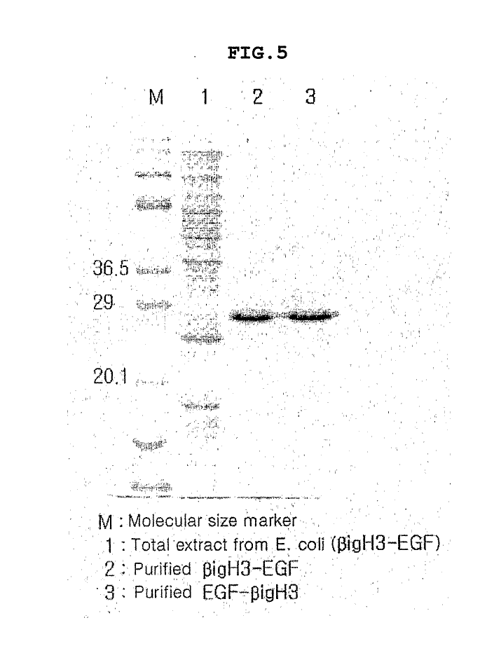

The supernatant was loaded on nickel-agarose column activated by binding buffer (20 mM phosphate, 0.5 M NaCl, 10 mM imidazole) to be passed through at the speed of 1-3 μm/min. The column was washed several times with the binding buffer, to which 20, 40, 60, 100, 300 and 500 mM imidazole solution (pH 7.4) was applied. Fusion protein was extracted from column, which was fractionated by 1 ml. The fractions were analyzed by 12% polyacrylamide gel electrophoresis to distinguish those fractions containing fusion protein (see FIG. 5).

### Example 7

### Confirmation of Fusion Protein

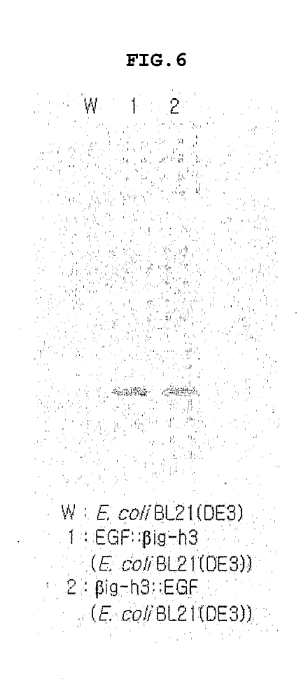

Polyacrylamide gel electrophoresis was performed with those fractions confirmed in Example 6 to have fusion protein. The protein band was transferred onto PVDF membrane, to which primary antibody (anti EGF-rabbit, 1:1000) was added, followed by incubation for one hour. Upon completion of incubation, the membrane was washed and then attached secondary antibody (rabbit-goat HRP, 1:1000), followed by further reaction for one more hour. The membrane was then washed. Upon completion of antibody adhesion, color reaction was induced by using 4-chloro-1-naphthol as a substrate to investigate the colored band with expected size of 26.5 kDa of EGF fused protein, leading to the confirmation of EFG fusion protein (see FIG. 6).

### Example 8

### Construction of a Vector for expressing βig-h3-EGF Fusion Nucleotide Sequence in a Plant

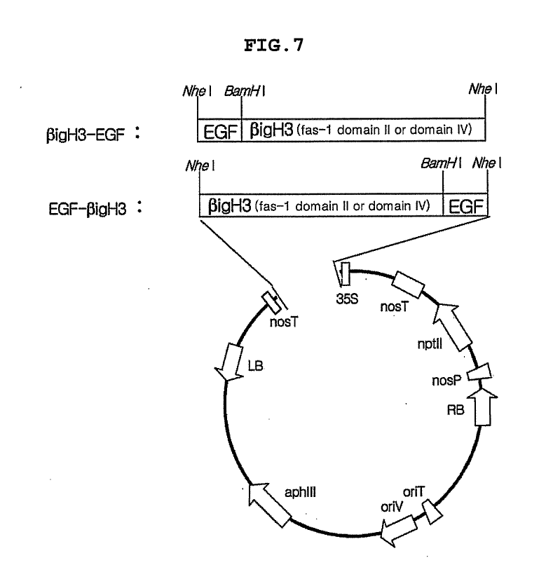

To subclone βig-h3-EGF fusion nucleotide sequence into plant expression cassette of binary vector pRD400 by PCR, two oligonucleotides were synthesized based on nucleotide sequences of βig-h3 and EGF of the present invention. Sense primer (5′-CTAGCTAGCGATGAAGTGGGTAACCTTTAT-3′), located in 5′ end of βig-h3 nucleotide sequence, was designed to have NheI restriction enzyme site and start codon of βig-h3 nucleotide sequence, while antisense primer (5′-CTAGCTAGCCGCAGTTCCCACCACTTAAGA-3′), located in 3′ end, was designed to have stop codon of EGF nucleotide sequence and NheI restriction enzyme site. PCR was performed as follows. 1.25 unit of Taq DNA polymerase (BM), 2.5 μl of 10× buffer, 2 μl of 2.5 mM dNTP, 0.25 μl of 100 pM primer, DNA containing 50 ng of gad nucleotide sequence and distilled water were mixed to make the final volume 25 μl. The prepared reacting solution was predenatured at 95° C. for 2 minutes, followed by 30 cycles of denaturation at 92° C. for 1 minute, annealing at 55° C. for 1 minute, polymerization at 72° C. for 1 minute, and final extension at 72° C. for 10 minutes (MJ Research, Inc. Minicycle™). The PCR product was stored at 4° C. for successful analysis, which was then electrophorezed on 0.8% TAE agarose gel. The corresponding band was extracted and target βig-h3-EGF DNA fragment was obtained. Purified βig-h3-EGF DNA fragment was digested with restriction enzyme NheI, which was inserted into binary vector pRD400 for plant expression, which had been digested with restriction enzyme XbaI. As a result, a vector for expressing βig-h3-EGF nucleotide sequence in a plant was constructed (see FIG. 7).

### Example 9

### Construction of a Vector for Expressing EGF-βig-h3 Fusion Nucleotide Sequence in a Plant

To subclone EGF-βig-h3 fusion nucleotide sequence into plant expression cassette of binary vector pRD400 by PCR, two oligonucleotides were synthesized based on nucleotide sequences of βig-h3 and EGF of the present invention. Sense primer (5′-CTAGCTAGCGATGAACAGCGATTCAGAATG-3′), located in 5′ end of EGF nucleotide sequence, was designed to have NheI restriction enzyme site and start codon of EGF nucleotide sequence, while antisense primer (5′-CTAGCTAGCCCGGTACGCGTAGAATCGAGA-3′), located in 3′ end of βig-h3 nucleotide sequence, was designed to have NheI restriction enzyme site and stop codon of βig-h3 nucleotide sequence. EGF-βig-h3 nucleotide sequence, obtained by the same manner as described in Example 8, was digested with restriction enzyme NheI, and then inserted into binary vector pRD400 for plant expression, which had already been digested with restriction enzyme XbaI, resulting in the construction of a vector for expressing EGF-βig-h3 nucleotide sequence in a plant (See FIG. 7).

### Example 10

### Plant Transformation

<10-1> Transfection of Agrobacterium tumefaciens GV3101

Agrobacterium tumefaciens GV3101 (mp90) (Plant-cell-rep. 15(11): 799-803 (1996)) was transfected with pRD400::(βig-h3-EGF) of Example 8 and pRD400::(EGF-βig-h3) of Example 9, respectively by conjugation. To select transfected Agrobacterium, the conjugated mixture was plated on solid LB medium supplemented with 50 mg/l of kanamycin and 30 mg/l of gentamycin, and cultured at 28° C. for 2 days. Then, transfected strains were selected. The selected Agrobacterium containing foreign gene was inoculated into super broth (BHI medium, pH 5.6), followed by culture at 28° C. for 2 days. The medium was ready to be used for transfection of a plant.

### <10-2> Melon Transformation

Sterilized melon seeds were sowed and seed leaves (cotyledons), in which growing points were eliminated, were obtained. In the meantime, Agrobacterium tumefaciens GV3101 (Mp90) (Plant-cell-rep. 15(11):799-803 (1996)) transfected with pRD400::(βig-h3-EGF) or pRD400::(EGF-βig-h3) was cultured in super broth (37 g/l Brain heart infusion broth (Difco) and 0.2% sucrose, pH 5.6) supplemented with 100 μM acetosyringone for 18 hours at 28° C. The culture solution was 20 fold-diluted with infecting medium. The infecting medium (pH 5.6) was composed of MSB5 (Murashige & Skoog medium including Gamborg B5 vitamins), 3.0% sucrose, 0.5 g/l of MES [2-(N-Morpholino)ethanesulfonic acid Monohydrate], 6.0 mg/l of kinetin, 1.5 mg/l of IAA (Indole-3-acetic acid), 1.0 mg/l of CuSO₄.5H₂O, 100 μM acetosyringone and 5% DMSO (dimethylsulfoxide).

The seed leaves were dipped in 40 ml of infecting medium, followed by culture for 20 minutes. Then, the seed leaves were transferred to co-culture medium (MSB5, 3.0% sucrose, 0.5 g/l of MES, 6.0 mg/l of kinetin, 1.5 mg/l of IAA, 1.0 mg/l of CuSO₄.5H₂O, 0.6% agar, 100 μM acetosyringone and 5% DMSO). Under the dark culture condition (26±1° C., 24 hours of night), the seed leaves were co-cultured for 3 days. The seed leaves were plated on selection medium (MSB5, 3.0% sucrose, 0.5 g/l of MES, 6.0 mg/l of kinetin, 1.5 mg/l of IAA, 1.0 mg/l of CuSO₄.5H₂O, 0.6% agar, pH 5.6, 100 mg/l of kanamycin and 500 mg/l of carbenicillin), followed by photo-cultivation for three weeks at 26±1° C. under the light condition of 16 light-hours with 4,000 Lux of illuminance, inducing shoots. The shoots were transplanted in rooting medium (MSB5, 3.0% sucrose, 0.5 g/l of MES, 0.1 mg/l of NAA (α-Naphthalene Acetic Acid), 1.0 Mg/l of CuSO₄.5H₂O, 0.6% agar, pH 5.6, 100 mg/l of kanamycin and 500 mg/l of carbenicillin), followed by culture for 2 weeks. The presumed transfected shoots were selected.

### <10-3> Cucumber Transformation

Sterilized cucumber seeds were sowed and seed leaves, in which growing points were eliminated, were obtained. In the meantime, Agrobacterium tumefaciens transfected with pRD400::(βig-h3-EGF) or pRD400::(EGF-βig-h3) was cultured under the same condition as described in Example <10-1>, and then cucumber seed leaf sections were dipped in infecting medium, prepared by mixing transfected Agrobacterium tumefaciens with the infecting medium having the same composition as that used in melon transformation in Example <10-2>, followed by mixing for 10 minutes.

The mixture was photo-cultivated in co-culture medium (MSB5 containing 2 mg/l of BAP and 0.01 mg/l of NAA) for two days at 26° C., and then Agrobacterium tumefaciens and the seed leaves were co-cultured for 4 days at 4° C. The seed leaves were further cultured in selection medium having the basic composition of MS-B5, 0.5 g/t of MES, 3% sucrose, and 0.4% phytagel and supplemented with 2 mg/l of BAP, 0.01 mg/l of NAA, 500 mg/l of carbenicillin, and 100 mg/l of kanamycin for 4 weeks at 26±1° C. with light condition of 16 hour-light/8 hour-dark with 8,000 Lux of illuminance. The regenerated shoots were transferred into rooting medium containing 0.01 mg/l of NAA, 100 mg/l of kanamycin and 0.4% agar, followed by culture at 26±1° C. with the light condition of 16 hour-light/8 hour-dark with 8,000 Lux of illuminance. The rooted plant was presumed as a transformant, which was analyzed by the method of Example 11.

### <10-4> Watermelon Transformation

Sterilized watermelon seeds were sowed and seed leaves, in which growing points were eliminated, were obtained. In the meantime, Agrobacterium tumefaciens transfected with pRD400::(βig-h3-EGF) or pRD400::(EGF-βig-h3) was cultured under the same condition as described in Example <10-1>, and then watermelon seed leaf sections were dipped in infecting medium, prepared by mixing transfected Agrobacterium tumefaciens with the infecting medium having the same composition as that used in melon transformation in Example <10-2>, followed by mixing for 10 minutes. The seed leafs were plated on co-culture medium (4.04 g/l of MSB5, 3.0% sucrose, 0.5 g/l of MES, 0.6% agar, pH 5.6) containing 2 mg/l of BAP, followed by further culture for 2 days at 25±1° C. with 16 hour-light condition with 4000 Lux of illuminance. The cultured seed leaves were plated on fresh medium (MSB5, 2 mg/l of BAP, 3.0% sucrose, 0.5 g/l of MES, 0.4% phytagel, pH 5.6, 500 mg/l of carbenicillin, 200 mg/l of kanamycin) and cultured for 4 weeks at 25±±1 to select shoots.

### <10-5> Rape Transformation

Sterilized rape seeds were sowed and leafstalks (petioles), in which growing points were eliminated, were obtained. In the meantime, Agrobacterium tumefaciens transfected with pRD400::(βig-h3-EGF) or pRD400::(EGF-βig-h3) was cultured under the same condition as described in Example <10-1>, and then rape leafstalks were dipped in infecting medium, prepared by mixing transfected Agrobacterium tumefaciens with the infecting medium having the same composition as that used in melon transformation in Example <10-2>, followed by mixing for 10 minutes. The leafstalks were cultured in co-culture medium (MSB5, 3% sucrose, 1 mg/l of 2,4-D, 6.5 g/l of agar powder, pH 5.8) at 25° C. for 2 days, and further cultured at 4° C. for 4 days. Regenerated shoots were transferred to selection medium (MSB5, 3% sucrose, 5 g/l of MES, 2 mg/l of BA, 0.01 mg/l of NAA, 20 mg/l of kanamycin, 500 mg/l of Pseudopen, 6.5 g/l of agar powder, pH 5.8) and cultured for two weeks at 25° C. under 16 hour-light/8 hour-dark condition to select transformed rape. Two weeks later, the shoots were transferred to another medium (pH 5.8) composed of MSB5, 3% sucrose, 5 g/l of MES, 0.1 mg/l of NAA, 20 mg/l of kanamycin, 500 mg/l of Psedopen, and 6.5 g/l agar to induce roots.

### <10-6> Tobacco Transformation

Sterilized tobacco seeds were sowed and young tobacco leaves, which were aseptic cultivated for more than two weeks, were obtained. In the meantime, Agrobacterium tumefaciens transfected with pRD400::(βig-h3-EGF) or pRD400::(EGF-βig-h3) was cultured under the same condition as described in Example <10-1>, then mixed with infecting medium having the same composition as that used for melon transformation of Example <10-2>. The obtained tobacco leaves were cut into pieces of 0.5-1 cm², which were dipped in the infecting medium for 10-15 minutes. The tobacco leaf sections were transferred to co-culture medium (MSB5, 3.0% sucrose, 0.5 g/l of MES, 1.0 mg/l of BAP, 0.1 mg/l of NAA, pH5.8, 0.6% agar), followed by co-cultivation for 2 days under dark culture condition (26±1° C., 24 hours night). The leaf sections were plated on selection medium (MSB5, 3.0% sucrose, 0.5 g/l of MES, 1.0 mg/l of BAP, 0.1 mg/l of NAA, 0.6% agar, pH5.6, 100 mg/l of kanamycin and 500 mg/l of carbenicillin), followed by photo-cultivation for 2 weeks at 26±1° C. with 4,000 Lux of illuminance under 16 hour light condition to induce shoot development. Shoots were transplanted to rooting medium (MSB5, 3.0% sucrose, 0.5 g/l of MES, 0.01 mg/l of NAA, 0.6% agar, pH 5.6, 100 mg/l of kanamycin and 500 mg/l of carbenicillin) and cultured for two weeks. The presumed transformed shoots with roots were selected.

### Example 11

### Confirmation of Transformant

Confirmation of transformants obtained in the Example 10 was performed as follows.

Plant genomic DNA was extracted from 10 mg of transfected shoots selected from selection medium by Edward's method (Nucleic Acids Research, 19:1349 (1991)), which was then used as a template for PCR.

The primers used for PCR with plant transformant of pRD400:: (βig-h3-EGF) were corresponding to the nucleotide sequence of βig-h3-EGF, that is, sense primer was 5′-CTAGCTAGCGATGAAGTGGGTAACCTTTAT′3′ and antisense primer was 5′-CTAGCTAGCCGCAGTTCCCACCACTGTAAGA-3′.

The primers used for PCR with plant transformant of pRD400::(EGF-βig-h3) were corresponding to the nucleotide sequence of EGF-βig-h3, that is, sense primer was 5′-CTAGCTAGCGATGAACAGCGATTCAGAATG-3′ and antisense primer was 5′-CTAGCTAGCCCGGTACGCGTAGAATCGAGA-3′.

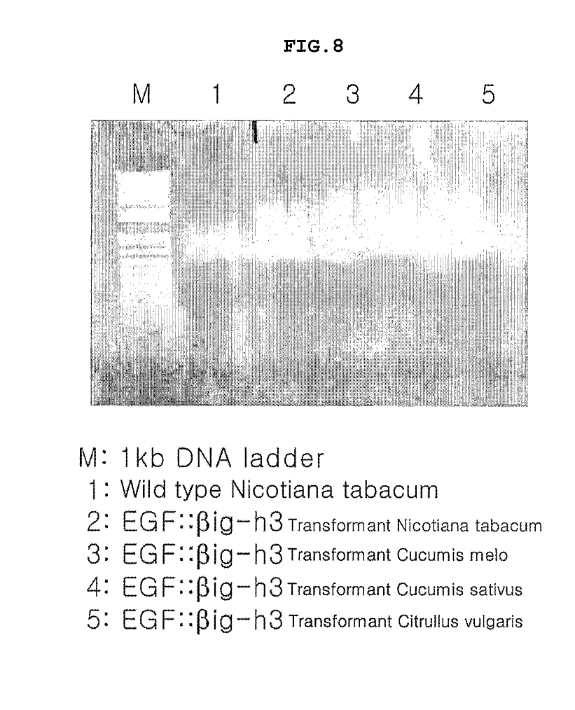

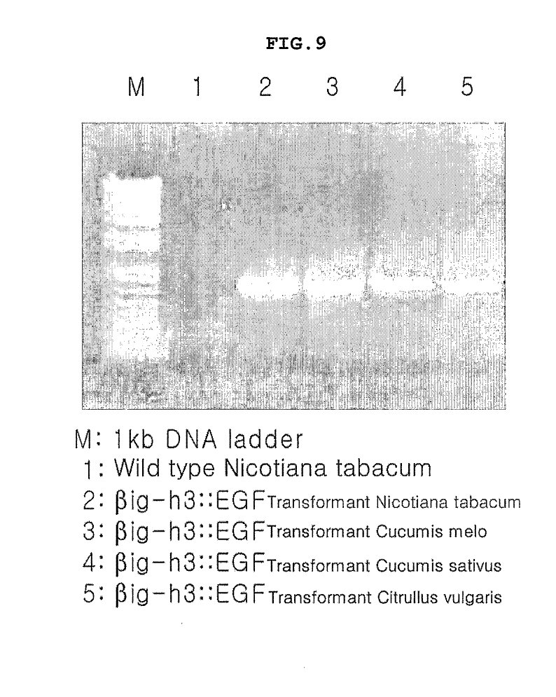

PCR was performed as follows; predenaturation at 96° C. for 2 minutes, denaturation at 94° C. for 1 minute, annealing at 55° C. for 1 minute, polymerization at 72° C. for 2 minute, 35 cycles from denaturation to polymerization, and final extension at 72° C. for 10 minutes. Tag polymerase was used for PCR. After PCR, the PCR product was analyzed on 1.0% agarose gel (See FIG. 8 and FIG. 9).

In FIG. 8, lane M is 1 kb DNA ladder, lane 1 is a PCR product produced by using chromosomal DNA of wild type tobacco, lanes 2, 3, 4 and 5 are PCR products produced by using each selected tobacco, melon, cucumber, and watermelon transformant DNAs as templates.

In FIG. 9, lane M is 1 kb DNA ladder, lane 1 is a PCR product produced by using chromosomal DNA of wild type tobacco, lanes 2, 3, 4 and 5 are PCR products produced by using each selected tobacco, melon, cucumber, and watermelon transformant DNAs as templates.

### Example 12

### Expression of Fusion Protein in Transformant

1 g of each transformed plant leaf was cut into pieces and put in a mortar, to which 2.5 ml of extraction buffer (5 ml of 100 mM Tris-Cl, pH 7.5, 40 ml of 500 mM EDTA, pH 8.0, 1.5 ml of 1 mg/ml leupeptin, 600 μl of 5 mg/ml BSA, 3 ml of 1 mg/ml DTT, added 50 μl of 30 mg/ml PMSF (stock: 0.003 g PMSF/10 μl of IPA) right before use) was added, followed by pulverization. The extract was centrifuged at 4° C. with 12,000 rpm for more than 30 minutes, and supernatant was obtained. The supernatant was transferred into a new tube, which was then stored in ice.

A dye (protein assay kit, Bio-Rad) was added to the above plant extract and OD₅₉₅ was measured with UV-spectrophotometer. The protein expressed in the transformed plant was quantified by comparing that of standard sample of bovine serum albumin by Bradford's method.

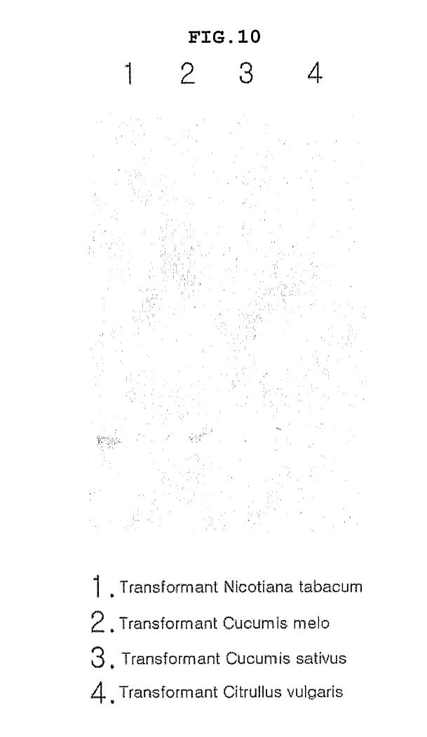

The supernatant obtained from the each plant transformant was adjusted to have equal amount of protein. Each sample was electrophorezed on 8% polyacrylamide gel to investigate the expression of fusion protein. Those samples confirmed to have fusion protein expressed therein were selected, followed by polyacrylamide gel electrophoresis. Protein band was transferred onto PVDF membrane. The primary antibody (anti EGF-rabbit, 1:1000) was loaded on PVDF membrane containing the protein, followed by incubation for one hour. Upon completion of the culture, the membrane was washed and the secondary antibody (rabbit-goat HRP, 1:1000) was loaded thereto, followed by another incubation for one hour. The membrane was washed. Upon completion of antibody adhesion, color reaction was induced by using 4-chloro-1-naphtol as a substrate to investigate the size of color band of EGF fusion protein, leading to the confirmation of the expression of EGF fusion protein (See FIG. 10).

### Example 13

### Cell Adhesion Activity of Fusion Protein Containing βig-h3 Fas-1 Domain

Human epidermal cells (HaCaT cells) used for cell adhesion activity test were cultured in DMEM (Gibco BRL) containing 10% fetal bovine serum, penicillin (100 U/ml), streptomycin (100 U/ml), and amphotericin (0.25 U/ml) in a 37° C. 5% CO₂ incubator.

Cell adhesion activity was measured as follows. First, fusion protein containing Fas-1 domain or other extracellular matrix proteins were adhered to a 96 well plate at 4° C. for 24 hours. The plate was washed twice with phosphate buffer to eliminate the remaining unattached proteins. To avoid non-specific reaction therein, each well was treated with phosphate buffer containing 2% BAS for one hour, then washed twice with phosphate buffer, followed by drying at room temperature. As an extracellular matrix protein herein, collagen and Regenin (βig-h3 domain IV, Regen Biotech Inc.) were used. Each cell was treated with trypsin and suspended in culture solution at the concentration of 2×10⁵ cells/ml, then distributed to each well of plate by 0.1 ml/well.

The well-plate stood for one hour at 37° C. The unattached cells were eliminated by washing with phosphate buffer. In the meantime, the attached cells were added with citrate buffer (pH 5.0) supplemented with 3.75 mM p-nitrophenol-N-acetyl-1-β-D-glycosaminide, acting as a hexosamidase substrate, and 0.25% triton X-100, followed by culture for one hour at 37° C. The enzyme activity in the mixture was terminated by adding 50 mM glycine buffer (pH 5.0) containing 5 mM EDTA. OD₄₅₀ was measured by using Multiscan MCC/340 microplate reader.

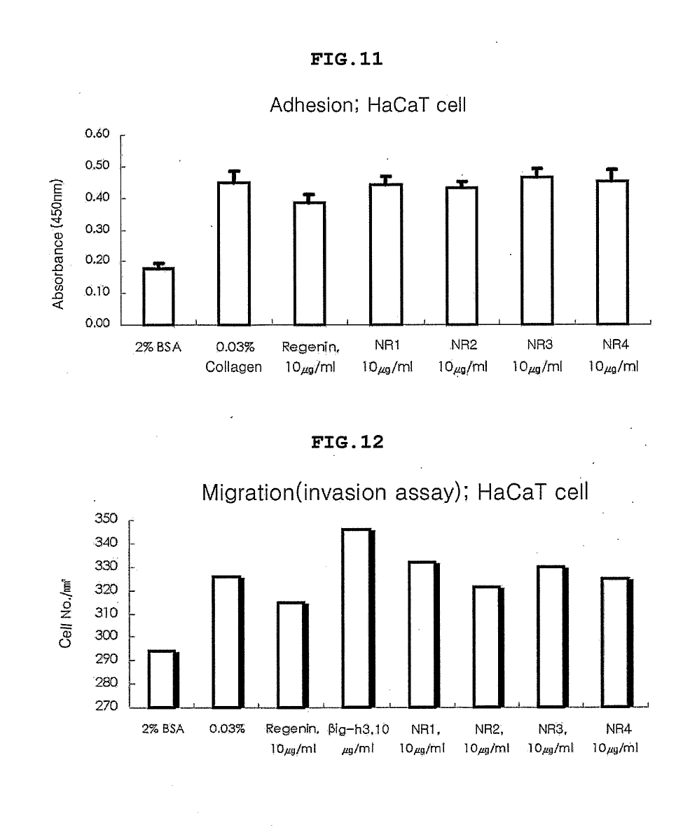

HaCaT cell adhesion activity was measured by using Regenin (βig-h3 domain IV), NR1 (EGF-Regenin), NR2 (his tag EGF-Regenin), NR3 (Regenin-EGF) and NR4 (EGF-albumin), and as a result, the adhesion activity was higher in fusion protein than in Regenin (βig-h3 domain IV) and was similar among fusion proteins (See FIG. 11).

### Example 14

### Cell Diffusion Activity by Fusion Protein Comprising βig-h3 Fas-1 Domain

To investigate whether or not such fusion proteins as NR1, NR2, NR3 and NR4 could maintain cell diffusion activity, one of the functions of a polypeptide comprising Fas-1 domain, invasion assay was performed by using HaCaT cell line.

Cell diffusion activity was measured as follows. First, fusion protein containing Fas-1 domain or other extracellular matrix proteins were attached to transwell polycarbonate membrane (pore size 8 μm, Corining Coaster, USA) for 24 hours at 4° C. Then, the membrane was washed twice with phosphate buffer to eliminate the remaining unattached proteins. To avoid non-specific reaction, each well was treated with phosphate buffer supplemented with 2% BAS for one hour, washed with phosphate buffer twice and dried at room temperature for 30 minutes. As an extracellular matrix protein herein, collagen, Regenin (βig-h3 domain IV, Regen Biotech Inc.) and βig-h3 were used. Each cell was treated with trypsin and suspended in culture medium at the concentration of 1.5×10⁶ cells/ml, then distributed to each well of plate by 0.2 ml/well. The cells were cultured for 6 hours in a 37° C. 5% CO₂ incubator. Cells attached inside wall of each well were eliminated and the membrane was fixed in 8% glutaraldehyde. Only the cells passing through the membrane were stained in 20% methanol solution containing 0.25% crystal violet, and the number of cells was counted by observing under microscope.

Cell diffusion activity was measured by using Regenin, collagen, βig-h3, NR1, NR2, NR3 and NR4. As a result, cell diffusion activity was not significantly different among fusion proteins, but the activity in fusion protein was proved to be improved from the level shown in Regenin and greatly increased than that in BSA, approaching to the level of collagen (See FIG. 12).

### Example 15

### Cell Proliferation Activity by Fusion Protein Comprising βig-h3 Fas-1 Domain

To confirm whether or not such fusion proteins as NR1, NR2, NR3 and NR4 could maintain the function of cell proliferation activity, one of major functions of EGF and a polypeptide comprising Fas-1 domain, cell proliferation test was performed with HaCaT cell line.

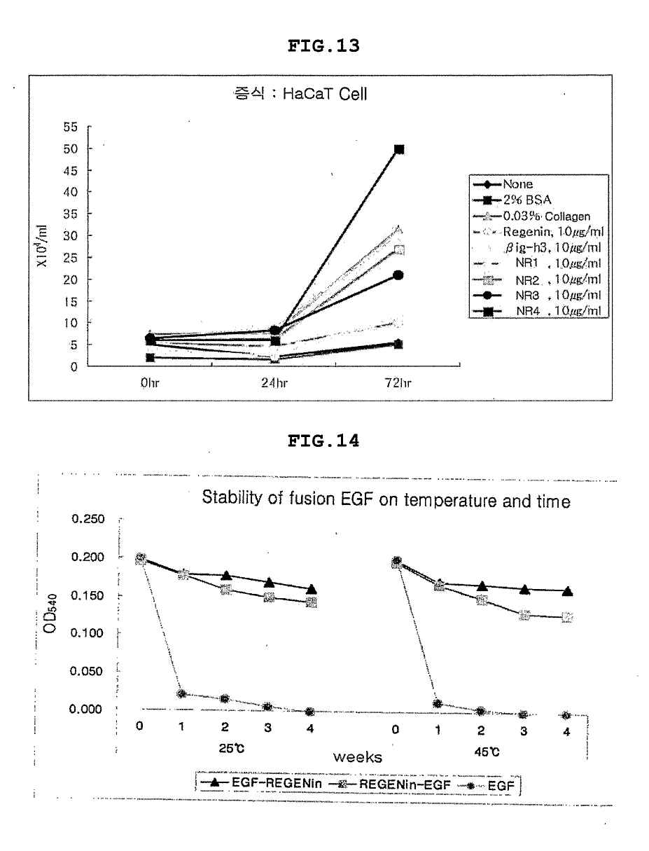

Each well was treated by the same manner as described in the above cell adhesion test. Same number of cells were loaded in the wells and cultured for 72 hours in a 37° C. 5% CO₂ incubator. The cells attached to the plate were taken off by using trypsin-EDTA at the time point of 0, 24 and 72 hour from the beginning of culture. The obtained cells were stained with trypan blue, and the number of stained cells was counted with hematocytometer. Cell proliferation activities in NR1, NR2, NR3 and NR4 were all similarly high or higher than that in collagen. In particular, cell proliferation activities in such fusion proteins were at least two times or up to 5 times higher than that in Regenin or βig-h3. And the highest cell proliferation activity was observed in NR4 (See FIG. 13).

### Example 16

### Investigation of Stability of Fusion Protein

The stabilities of EGF-βig-h3 and βig-h3-EGF fusion proteins separated from plant transformants in Example 12 were investigated.

EGF-βig-h3 and βig-h3-EGF fusion proteins and a control EGF (Sigma, USA) were diluted in 10 mM phosphate buffer, making the concentrations even. The diluted samples were stored for 4 weeks at each 25° C. and 45° C. Some of each sample was taken and remaining activities were investigated by ELISA once a week.

Mouse monoclonal recombinant human EGF IgG (R&D systems), a capture antibody, was diluted with 10 mM phosphate buffer at the ratio of 1:1000, which was distributed to a 96 well ELISA plate by 100 μl/well. The plate stood at room temperature (4-25° C.) for more than 8 hours. The capture antibody of each well was eliminated. The plate was washed with washing buffer (PBST; 10 mM phosphate buffer (pH 7.4), 0.05% Tween-20) three times. After adding stop solution by 300 μl/well, the plate stood at room temperature for 2 hours. Time dependent samples and EGF standard proteins (KOMA, Korea) diluted by different concentrations were distributed to the plate (100 μl/well) prepared like the above, which was stored at room temperature or at 4° C. for more than 8 hours. Later, the plate was washed with washing buffer three times. Detection antibody (1:1000) was added to the plate by 100 μl/well, followed by reaction for two hours. After washing with washing buffer three times again, 1:50 diluted streptavidin-HRP (streptavidin horse radish phosphatase) was added to each well by 100 μl, which was left for one hour, then washed by the same procedure as described above. Substrate buffer (coloring reagent A (H₂O₂) and coloring reagent B (Tetramethylbenzidine) were mixed at the ratio of 1:1, R&D Systems) was diluted with phosphate buffer at the ratio of 1:4, and the diluted solution was added to each well, followed by observation of color (blue) development. By observing the level of color development, stop solution was added to terminate (yellow) the reaction. OD₅₄₀-OD₅₇₀ was measured within 30 minutes after adding stop solution, which was compared with the value of standard protein to calculate the relative concentration of each sample. That is, time-dependent remaining activity of EGF at different temperatures was measured to evaluate the stability of fusion protein (See FIG. 14).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

### INDUSTRIAL APPLICABILITY

The present invention provides human EGF with improved stability and enhanced functions by fusing a polypeptide comprising Fas-1 domain having the activities of cell adhesion and wound healing to human EGF.

1. A fusion protein, in which a polypeptide comprising Fas-1 domain is fused in frame to N-terminal or C-terminal of human EGF.

2. The fusion protein of claim 1, wherein said Fas-1 domain is selected from the group consisting of polypeptides containing Fas-1 domain I, II, III and IV of human βig-h3 protein.

3. A nucleotide sequence encoding the fusion protein in which a polypeptide comprising Fas-1 domain is fused in frame to N-terminal or C-terminal of human EGF.

4. The nucleotide sequence of claim 3, wherein said nucleotide sequence encoding the human EGF contains a sequence represented by SEQ. ID. No 1.

5. The nucleotide sequence of claim 3, wherein said nucleotide sequence encoding the Fas-1 domain is characteristically the nucleotide sequence encoding a polypeptide selected from the group consisting of polypeptides containing Fas-1 domain I, II, III and IV of human βig-h3 protein.

6. The nucleotide sequence of claim 3, wherein said nucleotide sequence encoding the Fas-1 domain contains a sequence represented by SEQ. ID. No 1 or No 2

7. An expression vector containing any nucleotide sequence of claim 3 to claim 6.

8. A transformant transformed by the expression vector of claim 7.

9. The transformant of claim 8, wherein said transformant is a plant.

10. A method for producing human EGF, which comprises the steps of preparing the transformant of claim 8 and obtaining the expressed protein therein.

11. The method for producing human EGF of claim 10, wherein said transformant is a plant.

12. A method for improving stability of a peptide or a protein, which is characterized by improving stability of a peptide or a protein which is unstable as being expressed after the fusion with a polypeptide comprising Fas-1 domain.

13. The method for improving stability of claim 12, wherein said peptide or the protein is human EGF peptide or protein.

14. A method for enhancing functions of human EGF, which characteristically enhancing functions of human EGF by fusing a polypeptide comprising Fas-1 domain to human EGF.


SEQUENCE LISTING (3 sequences)

SEQ ID NO: 1 - DNA, 162 bp, Artificial Sequence
  Optimize nucleotide sequence encoding hEGF
atgaacagcgattcagaatgtccactgagccatgacggatactgcctgcacgacggcgtctgcatgtacatcgaggcactggacaagtacgcgtgcaactgtgttgttggatacatcggtgagcgttgtcaataccgtgatcttaagtggtgggaactgcgc

SEQ ID NO: 2 - DNA, 399 bp, Artificial Sequence
  Optimized nucleotide sequence encoding fas-1 domain II
atgactaacaacatacaacaaattattgaaattgaagacactttcgaaacgcttagggctgcagttgcagcgtctgggttaaataccatgttggagggtaatggacagtacaccttgctggctcctacaaacgaggcttttgaaaagataccctcagagacccttaacagaatcttgggcgatccagaagccctccgtgaccttttgaataaccacattcttaagtccgcaatgtgtgccgaggccattgtggccggattgagcgtagaaactctagagggaactacattagaggttggatgcagtggtgatatgcttaccataaatggcaaagctatcatctctaataaagatatcctcgctacaaacggtgtcattcattatatcgacgagctcctg

SEQ ID NO: 3 - DNA, 519 bp, Artificial Sequence
  Optimized nucleotide sequence encoding fas-1 domain IV
atgaaagaaactgctgccgcaaagttcgaaagagagcatatggactctccagatttaggaaccctcgttcccaggggcagtatggccgatatcctcaccccaccgatgggtacggtcatggacgtactgaagggcgataaccgtttctcaatgttggtggctgctatccaatctgctggtttaacagaaacccttaatcgcgagggggtttacacagtctttgcgcccacaaacgaggcctttcgagcattgcctccaagagagaggagccgtttgcttggtgacgcaaaggagcttgctaacattcttaagtatcatattggagatgagattctcgtttccgggggaattggtgctctcgtgcgtttaaaatctctccaaggtgacaagcttgaagtatctctcaagaacaacgttgtgagcgtgaacaaagaaccagttgctgaacctgacatcatggcaactaatggagtcgtgcacgttataactaatgtattgcaacctccagccaatcttgaa